(19) United States
(12) Patent Application Publication
(10) Pub. No.: US 2006/0189866 A1
(43) Pub. Date: Aug. 24, 2006

(54) System for image-guided pulsed magnetic field diagnosis and treatment

(75) Inventors: Alex W. Thomas, London (CA); Frank S. Prato, London (CA); Jeff D. Winter, Barrie (CA); Terry R. Thompson, London (CA); Cheryl R. McCreary, Balzac (CA)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(73) Assignee: Fralex Therapeutics, Inc.

(21) Appl. No.: 11/320,194

(22) Filed: Dec. 27, 2005

Related U.S. Application Data
(63) Continuation of application No. PCT/CA04/00945, filed on Jun. 25, 2004.
(60) Provisional application No. 60/482,709, filed on Jun. 27, 2003.

(51) Int. Cl. A61B 5/055

(52) U.S. Cl. 600/411

(57) ABSTRACT

A method and apparatus comprising image-guided application of a pulsed magnetic field for the diagnosis and/or treatment of various physiological, neurological and/or behavioral pathologies or conditions.

### CROSS-REFERENCE TO RELATED APPLICATIONS.

This Application is a continuation under 35 U.S.C. § 365(c) and 35 U.S.C. § 120 of International Application No. PCT/CA 2004/000945, filed Jun. 25, 2004, which is incorporated by reference in its entirety. This application also claims priority under 35 U.S.C. § 119 of U.S. Provisional Application Ser. No. 60/482,709, filed Jun. 27, 2003, which is incorporated by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The present invention relates to magnetic fields and in particular, to the use of image-guided application of a pulsed magnetic field for the diagnosis and/or treatment of various physiological, neurological and/or behavioral pathologies or conditions. (2) Description of Related Art, Including Information Disclosed Under 37 C.F.R. §§ 1.97 and 1.98.

Diverse studies have shown that the behavioral, cellular and physiological functions of animals can be affected by magnetic stimuli. Weak magnetic fields exert a variety of biological effects ranging from alterations in cellular ion flux to modifications of animal orientation and learning, and therapeutic actions in humans. A number of magnetic field exposures have been shown to reduce exogenous opiate (e.g., morphine) and endogenous opioid peptide (e.g., endorphin) mediated analgesia in various species, including humans (Kavaliers, M. and Ossenkopp, K. -P. (1991) Opioid systems and magnetic field effects in the land snail, Cepaea nemoralis. Biol. Bull. 180: 301-309; Prato, F. S. , Ossenkopp, K -P., Kavaliers, M., Sestini, E. A. , and Teskey, G. C. (1987) Attenuation of morphine-induced analgesia in mice by exposure to magnetic resonance imaging: Separate effects of the static, radio-frequency and time-varying magnetic fields. Mag. Res. Imag. 5, 9-14; Betancur, C., Dell'Omo, G. and Alleva E., (1994) Magnetic field effects on stress-induced analgesia in mice: modulation by light, Neurosci. Lett., 182 147-150; Kavaliers, M., Ossenkopp, K-P., Prato, F. S. , and Carson, J. (1994) Opioid systems and the biological effects of magnetic fields. In Frey A H (ed): On the nature of electromagnetic field interactions with biological systems; Austin, RG Landis Co. pp.181-190; Del Seppia, C., Ghione, S., Luchi, P., and Papi, F. (1995) Exposure to oscillating magnetic fields influences sensitivity to electrical stimuli. 1: Experiments on pigeons. Bioelectromagnetics 16:290-294; Papi, F., Ghiopne, S., Rosa, C., Del Seppia, C. and Luschi, P. (1995) Exposure to oscillating magnetic fields influences sensitivity to electrical stimuli 11: Experiments on humans. Bioelectromagnetics. 16:295-300). As well, extremely low frequency (ELF) magnetic field exposures are reported to modify homing pigeon behavior (Papi, F., Luschi, P. and Limonta, P. (1991) Orientation-disturbing magnetic treatment affects the pigeon opioid system. J. Exp. Biol. 160, 169-179) and spatial learning in rodents (Kavaliers, M., Eckel, L. A. & Ossenkopp, K -P (1993) Brief exposure to 60 Hz magnetic fields improves sexually dimorphic spatial learning performance in the meadow vole, Microtus pennsvivanicus. J comp. Physiol. A. 173, 2341-248 and Kavaliers, M., Ossenkopp, K -P., Prato, F. S. et al. (1996) Spatial learning in deer mice: sex differences and the effects of endogenous opioids and 60 Hz magnetic fields. J comp. Physiol A (In press)) in a manner consistent with alterations in opioid function.

There are several theories addressing the mechanism of the effect of low frequency magnetic field exposure on tissues. For example, low frequency magnetic field exposures have been proposed to exert their effect(s) through the induction of electric currents (Polk, C. (1992) Dosimetry of extremely low frequency magnetic fields. Bioelectromagnetics Supp. 1, 209-235; Weaver, J. S. and Astumian, R. D. (1990). The response of living cells to very weak electric fields; the thermal noise limit. Science, Wash. 247, 459-462). Weak magnetic fields have also been proposed to be detected by particles of magnetite in tissue and by virtue of this detection have a physiological effect (Kirschvink, J. L. and Walker, M. M. (1985). Particle size considerations for magnetite-based magnetoreceptors. In Magnetite biomineralization and magnetoreception in organisms: a new biomagnetism (ed. J. L. Kirschvink, D. S. Johnes & B. J. MacFadden), pp. 243-256. New York: Plenum Press); however, this magnetite based mechanism is not widely believed (Prato, F. S., Kavaliers, M. and Carson, J. J. L. (1996) Behavioral evidence that magnetic field effects in the land snail, Cepaea nemoralis, might not depend on magnetite or induced electric currents. Bioelectromagnetics 17, 123-130).

Extremely low frequency (ELF) magnetic fields are a physical agent, which have little attenuation in tissue and, therefore, can be used to alter endogenous processes provided they can be detected and their detection can be coupled to a physiological process. It is now shown that magnetic fields may be designed as time varying signals such that they can be used to alter specific targeted physiological processes and in this manner can be used to treat/modify various neurological and physiological conditions and behaviors.

U.S. Pat. No. 6,234,953, the subject matter of which is hereby incorporated herein by reference, describes the use of specific complex low frequency pulsed magnetic fields (Cnps) for the treatment of various physiological, neurologicaland/or behavioral pathologies or conditions, including pain, anxiety, and depression.

While complex low frequency pulsed magnetic fields (Cnps) are useful in treating various physiological, neurological and/or behavioral pathologies or conditions, it is desirable to improve the effectiveness of using Cnps for diagnosis and treatment of various pathologies or conditions.

### BRIEF SUMMARY OF THE INVENTION

The present invention relates to a method, system and use of image-guided application of a pulsed magnetic field for the diagnosis and/or treatment of various physiological, neurologicaland/or behavioral pathologies or conditions.

In one aspect of the present invention, there is provided a method for treatment and/or diagnosis of a physiological, neurological and/or behavioral pathology or condition in a subject, the method comprising:
applying a pulsed magnetic field to a targeted area in the subject, in combination with imaging the targeted area to verify the effectiveness of the pulsed magnetic field.

In another aspect of the present invention, there is provided a method that utilizes image-guided therapeutic application of magnetic fields, wherein specific pulsed magnetic fields functionally activate metabolic and molecular processes in the brain to diagnose physiological, neurological and/or behavioral pathologies or conditions.

In another aspect of the present invention, there is provided a method that utilizes image-guided therapeutic application of magnetic fields, wherein specific pulsed magnetic fields functionally inhibit metabolic and molecular processes in the brain, which, for example, can be applied to treat pain or anxiety.

In yet another aspect of the present invention, treatment and diagnosis can be guided to targeted areas of the brain, or any other targeted tissue areas.

In another aspect of the present invention, alterations in brain function is visualized and validated through functional, anatomical, and/or molecular imaging techniques.

In another aspect of the present invention, efficacy of treatment and alleviation of symptoms is monitorable.

In yet another aspect of the present invention, there is provided a method that customizes the application of specific pulsed magnetic fields to individuals for the treatment of neurological disorders or symptoms like pain, anxiety or depression, permitting development and evaluation of treatment on an individual basis through the imaging of specific targets.

In another aspect of the invention, the image-guided application of the pulsed magnetic field is used to monitor the effect of the magnetic field on various physiological, neurological and/or behavioral pathologies or conditions.

In another aspect of the present invention, the effect is monitored using molecular, functional, and/or anatomical medical imaging devices.

In another aspect of the present invention, the pulsed magnetic field is generated using magnetic field gradients and/or a radio frequency transmitter in clinical and research magnetic resonance imaging (MRI) devices and the imaging device is the MRI device.

In yet another aspect of the present invention, the imaging device is a positron emission tomography (PET) device or a single photon emission computerized tomography (SPECT) device. An independent device generates the pulsed magnetic field.

In yet another aspect of the present invention, the image-guided application of the pulsed magnetic field is used to select pulsed magnetic field parameters to optimize their effectiveness in producing various physiological, neurological and/or behavioral responses.

In yet another aspect of the present invention, the image-guided application of the pulsed magnetic field is achieved using an MRI device.

In another aspect of the present invention, an MRI device is used to treat physiological, neurological and/or behavioral pathologies or conditions while a patient or volunteer is having a diagnostic imaging procedure. In particular, claustrophobia or anxiety may be treated.

In still another aspect of the present invention, the pulsed magnetic field is used to emphasize image contrast. For example, the stimulation of pain centers allows visualization of opioid receptor activity.

In accordance with another aspect of the present invention, there is provided a method for the diagnosis of a physiological, neurological and/or behavioral condition in a subject, the method comprising: applying a specific low frequency pulsed magnetic field (Cnps) to a target tissue of the subject to initiate a physiological, neurological and/or behavioral response; and imaging the target tissue to monitor a physiological, neurological and/or behavioral function in order to determine the physiological, neurological and/or behavioral condition of the subject. The steps of applying and imaging may be simultaneous.

In accordance with another aspect of the present invention, there is provided a method for the diagnosis of disease conditions in a subject, the method comprising: exposing a subject to Cnps within a functional and/or molecular imaging apparatus for a time effective to produce a physiological response; monitoring a selected physiological function with functional and/or molecular imaging; evaluating a change in the selected physiological function with functional and/or molecular imaging; assessing the change in the selected physiological function with functional and/or molecular imaging; and classifying the subject into a disease category based on the assessment of the change in the selected physiological function.

In accordance with another aspect of the present invention, there is provided a method for the diagnosis of disease conditions in a subject, the method comprising: exposing a subject simultaneously to a selected Cnps and a functional and/or molecular imaging technique while monitoring a selected physiological function; evaluating any change in the selected physiological function; assessing the change in the selected physiological function; and classifying the subject into a disease category based on the assessment of the change in the selected physiological function.

In accordance with another aspect of the present invention, there is provided a method for the treatment of a physiological, neurological and/or behavioral condition in a subject, the method comprising: applying a specific low frequency pulsed magnetic field (Cnps) to a target tissue of the subject; imaging the target tissue of the subject; and repeating application of the specific low frequency pulsed magnetic field (Cnps) and imaging until sufficient treatment of the condition is attained. The steps of applying and imaging may be simultaneous.

In accordance with another aspect of the present invention, there is provided a method for the treatment of a physiological, neurological and/or behavioral condition in a subject, the method comprising: applying a specific low frequency pulsed magnetic field (Cnps) to a target tissue of the subject; imaging the target tissue of the subject; optimizing the Cnps based on imaging; and repeating application of the optimized Cnps and imaging until sufficient treatment of the condition is attained. The steps of applying and imaging may be simultaneous.

In accordance with another aspect of the present invention, there is provided a method for the treatment of a physiological, neurological and/or behavioral condition in a subject, the method comprising: imaging a target tissue of the subject; identifying an activation pattern of the target tissue; applying a specific low frequency pulsed magnetic field (Cnps) to the target tissue; imaging the target tissue of the subject; and repeating application of the specific low frequency pulsed magnetic field (Cnps) and imaging until a sufficiently modified activation pattern is attained. The steps of applying and imaging may be simultaneous.

In accordance with another aspect of the present invention, there is provided a method for the treatment of a physiological, neurological and/or behavioral condition in a subject, the method comprising: imaging a target tissue of the subject; identifying an activation pattern of the target tissue; applying a specific low frequency pulsed magnetic field (Cnps) to the target tissue; imaging the target tissue of the subject; optimizing the Cnps based on imaging, and repeating application of the optimized Cnps and imaging until a sufficiently modified activation pattern is attained. The steps of applying and imaging may be simultaneous.

In accordance with another aspect of the present invention, there is provided a use of an image-guided application of a pulsed magnetic field to diagnose and/or treat a physiological, neurological and/or behavioral condition.

In accordance with another aspect of the present invention, there is provided an electrotherapy system for treatment and/or diagnosis of a physiological, neurological and/or behavioral pathology or condition in a subject, the system comprising an imaging device and at least one pulsed magnetic field generating member, wherein the system provides application of a pulsed magnetic field from the at least one pulsed magnetic field generating member to a targeted area in the subject, in combination with imaging the targeted area with the imaging device to verify effectiveness of the pulsed magnetic field.

### BRIEF DESCRIPTION OF THE SEVERAL VIEWS OF THE DRAWING(S)

The present invention will become more fully understood from the detailed description given herein and from the accompanying drawings, which are given by way of illustration only and do not limit the intended scope of the invention.

FIG. 1 shows preliminary MRI images of brain activation due to a specific low frequency pulsed magnetic field gradient;

FIG. 2a shows an increase in the activation of pain centers in the brain for an individual responding to a thermal stimulus on their non-dominant right hand;

FIG. 2b shows the effect of applying a specific pulsed magnetic field, whereby there is a decrease in the activation of pain centers in the brain for the same individual shown in FIG. 2a responding to the same thermal stimulus; and

FIG. 3 is a scheme showing an embodiment of a method of the present invention.

### DETAILED DESCRIPTION OF THE INVENTION

Specific complex pulsed magnetic fields (Cnps) may be effectively used to treat physiological, neurological and/or behavioral disorders including, but not limited to pain, anxiety, and depression. Applicants have now developed a new method and system to verify the effectiveness of a pulsed magnetic field for treatment and/or diagnosis.

In one embodiment, the pulsed magnetic field is applied to the targeted area(s) and an image of the targeted area(s) is taken using an imaging device to verify the effectiveness of the pulsed magnetic field. Typically, to verify the effectiveness of the pulsed magnetic field, a contrast in the image is observed, as described more fully below with respect to the FIGURES. If the desired contrast in the image is not obtained, the pulsed magnetic field is modified and re-applied until the desired contrast is achieved.

The application of a pulsed magnetic field, in combination with imaging to verify the effectiveness of the pulsed magnetic field, is referred to as image-guided application of magnetic fields.

Image-guided therapeutic application of magnetic fields is used in various embodiments of the invention to functionally activate metabolic and molecular processes in the brain and other targeted areas using specific pulsed magnetic fields to diagnose physiological, neurological and/or behavioral pathologies or conditions. For instance, the pulsed magnetic fields can be used to activate pain (e.g., stimulate pain centers) in targeted area(s), which correlates with a contrast in the images of the targeted area(s), which allows visualization of opioid receptor activity. The degree of activation of pain with their location will allow differential diagnosis, which can guide the treatment.

Image-guided therapeutic application of magnetic fields is used in various embodiments of the invention to functionally inhibit metabolic and molecular processes in the brain and other targeted areas, which, for example, can be applied to treat pain or anxiety. Image-guided therapeutic application of this type can be used in combination with an MRI device to treat claustrophobia or anxiety while a patient or volunteer is having a diagnostic imaging procedure.

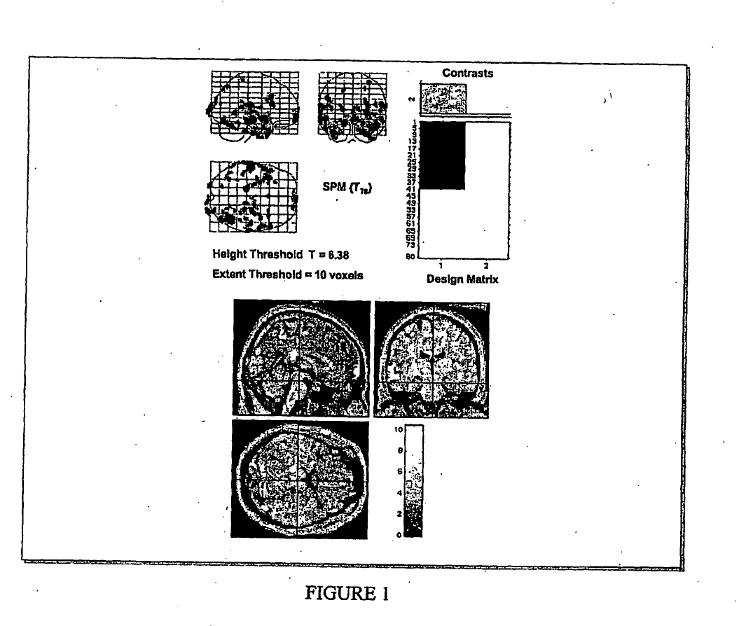

The effects of magnetic fields can be visualized using molecular, functional, and/or anatomical medical imaging devices, such as MRIs. For instance, FIG. 1 shows preliminary MRI images of brain activation due to a specific low frequency pulsed magnetic field gradient. Therefore, relatively weak pulsed magnetic fields may be used diagnostically or therapeutically in a conventional imaging device.

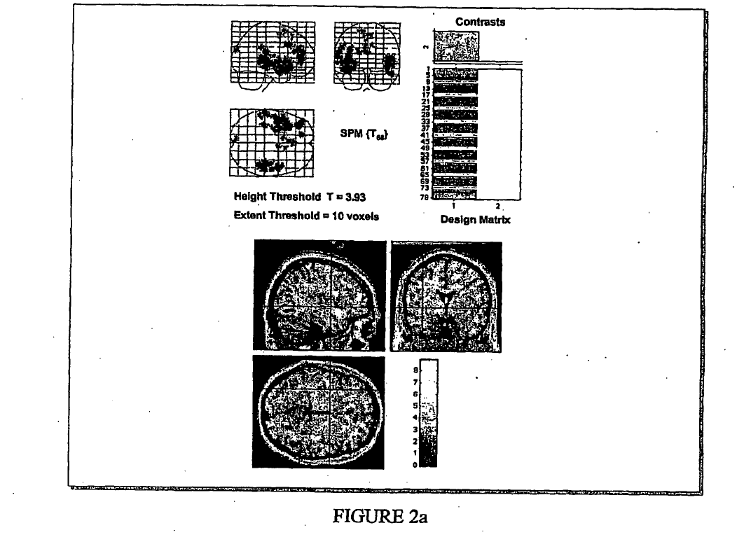

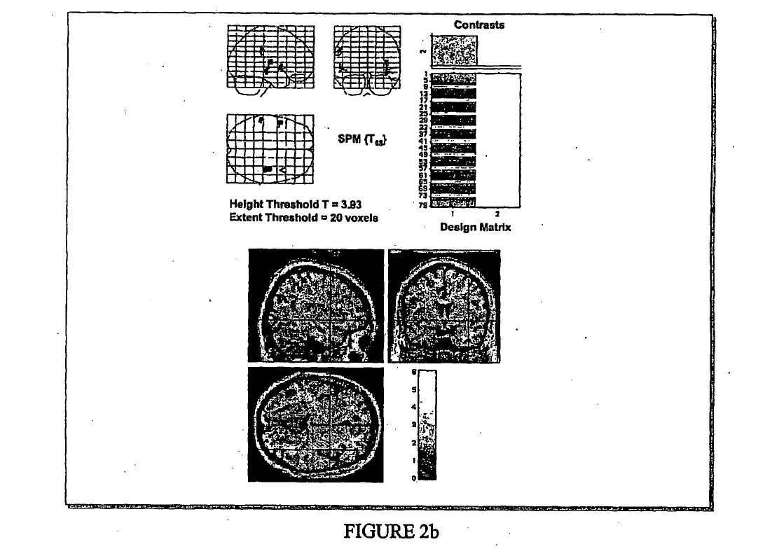

In another embodiment, FIG. 2a shows an increase in the activation of pain centers in the brain for an individual responding to a thermal stimulus on their non-dominant right hand. FIG. 2b shows the effect of applying a specific pulsed magnetic field, whereby there is a decrease in the activation of pain centers in the brain for the same individual shown in FIG. 2a responding to the same thermal stimulus. For instance, the images of FIG. 2b show a decrease in contrast compared to the images of FIG. 2a, verifying the effectiveness of the specific pulsed magnetic field. If such a response was not apparent in the image of FIG. 2b, the magnetic pulse is modified and reapplied. An image is taken, either after application of the pulse or simultaneously, which verifies the effectiveness of the specific pulsed magnetic field. The steps are repeated until the desired effect is achieved, a decrease in contrast of the image.

The specific pulsed magnetic fields of the present invention are capable of functionally activating metabolic and molecular processes in the brain and other targeted areas. In some embodiments, the pulsed magnetic filed may be generated using magnetic field gradients and/or a radio frequency transmitter in clinical and research magnetic resonance imaging (MRI) devices.

The specific pulsed magnetic fields may be comprised of a plurality of intermittent waveforms. The waveform is designed to look like the corresponding electromagnetic waveform of the target tissue. For example, if the target tissue were a part, or parts, of the brain then the waveform would correspond to the energetic activity of those parts. If an electroencephalogram (EEG) could record that activity, then the waveform would mimic the EEG, as exemplified in U.S. Pat. No. 6,234,953, the subject matter of which is hereby incorporated by reference.

After each waveform, or between successive waveforms, there is a delay referred to as a latency period. This delay is progressively set to increase, or decrease, in length with time. This effectively modulates, in time, the frequency of appearance of the waveform. The specific lengths and progression of the Cnp waveforms are related to the target tissue. With respect to the central nervous system (CNS), for example, there are a number of characteristic frequencies which relate to: a) frequencies specific to the area of the brain; b) frequencies associated with communication/connection between different brain regions; and c) frequencies and phase offsets associated with the coordination of different brain regions for a specific function. Now, although the waveform has been designed to stimulate neuronal activity for a specific region, electrical activity of a region of the CNS will vary between individuals, and over time, within an individual. Therefore, to target a function, the frequency of presentation of the waveform should match the frequency of the target. However, the target is varying within a frequency bandwidth. These CNS frequencies vary between approximately 7 Hz to 300 Hz. (For example: 7 Hz corresponds to alpha rhythm; 10 Hz thalamic activity; 15 Hz autonomic time; 30 Hz intralaminar thalamus and temporal regions associated with memory and consciousness; 40 Hz connection between hippocampal and amygdal temporal regions; 45 Hz hippocampal endogenous frequency; 80 Hz hippocampal-thalamic communication; 300 Hz motor control.) These frequencies have upper limits due to neuronal electrical properties, that is: after a neuron “fires” it is left in a hyperpolarized state and cannot fire again until it recovers.

To change the electrical activity of the target tissue in the CNS, the Cnp must “latch on” or more appropriately, entrain, to the appropriate frequency and either slow it down or speed it up. The waveform itself does not change substantially, rather, the frequency discussed herein corresponds to the rate at which the waveform is presented and the rate at which electrical spikes occur in the target tissue. Generally, for the CNS, as the frequency of neuronal activity is increased the amount of tissue involved per burst of activity decreases. Conversely, as the frequency is decreased a greater amount of tissue is synchronized and recruited throughout the CNS. For example, a) greater speed of cognitive processing can be associated with increased rates; b) if the rate is decreased significantly in humans or animals with epileptic-type disorders so much tissue can be recruited that seizures will occur. Therefore, the ramping up or ramping down of the rate of presentation of the waveform will: a) ensure that at least at some time the applied and endogenous rates will be matched (provided of course that the initial rate is greater than the endogenous if the purpose is to reduce the endogenous rate or lower if the purpose is to increase the endogenous rate); and b) “pull down” or “push up” the endogenous rate.

As a result of the application of the Cnp the synchrony of the electrical activity of the target can be disrupted. Before the application of another Cnp can be effectual the tissue must recover its synchrony. It is allowed to do so by providing a refractory period between application of successive Cnps where the length of the refractory period is determined by the target. For example, if the Cnps are applied to a target in humans that is associated with “awareness,” then the target will recover only after the awareness anticipation time is exceeded (e.g., 1200 ms). Another example would be the application for the same target, but in rodents without significant awareness, in which case the refractory period could be reduced to 400 ms. If the Cnps are to be applied for long periods of time per day, e.g., hours, then the refractory periods should be increased to 10 seconds to avoid possible immunosuppression. Immunosuppression has been shown to occur when the CNS is stimulated chronically and this may be minimized if the refractory periods of this stimulation are increased to more than 7 seconds. It must be pointed out that the Cnp features are related to the underlying physiology and that endogenous frequencies vary between individuals and within an individual. Therefore, there is tolerance on the feature specifications for any Cnp designed for a specific target. However, image-guided magnetic therapy will allow the Cnp parameters to be customized to the individual patient/subject and target tissue. For instance, to optimize the pulsed magnetic field parameters for pain therapy, the pain centers associated with pain control are activated or inhibited, as deduced from the image taken of the brain. If the pain centers are not optimally affected, as deduced from the image taken of the brain, then the parameters of the pulsed magnetic field are modified and the imaging repeated to achieve optimization.

The pulsed magnetic fields may be generated using a variety of electrotherapy systems in order to treat and/or diagnose a physiological, neurological and/or behavioral pathology or condition. The electrotherapy system may have an imaging device and at least one pulsed magnetic field generating member, such as a tube and/or coil, more typically, a gradient tube and/or gradient coil. In one embodiment of an electrotherapy system, two sets of volume coils for each of the three dimensions are used. One set would produce the DC offset, e.g., Helmholtz configuration. The second would be used to define magnetic field gradients, e.g., Maxwell configuration. (Prato, F. S. , Kavaliers, M. & Carson, J. J. L. (1996a) Behavioral evidence that magnetic field effects in the land snail, Cepaea nemoralis, might not depend on magnetite or induced electric currents. Bioelectromagnetics. 17, 123-130; Kavaliers, M., Ossenkopp, K -P., Prato, F. S. et al. (1996) Spatial learning in deer mice: sex differences and the effects of endogenous opioids and 60 Hz magnetic fields. J comp. Physiol A (In the press); Prato, F. S. ; Kavaliers, M.; Carson, J. L. L. (1996) Behavioral evidence that magnetic field effects in the land snail, Cepaea nemoralis, might not depend on magnetite or induced electric currents. Bioelectromagnetics. 17:123-130.) This type of electrotherapy system would be ideal for acute and chronic exposures in which the subject can stay in one position, e.g., treatment of pain while the subject is in bed. For mobile subjects, delivery would typically be through the use of surface coils either singly, as on the surface of the body, or around the neck or as a Helmholtz pair placed on either side of the knee.

The image devices used in the present invention may be selected from a variety of imaging devices such as MRI devices, positron emission tomography (PET) devices, single photon emission computerized tomography (SPECT) devices and the like. The pulsed magnetic field may or may not be generated independently of the imaging devices.

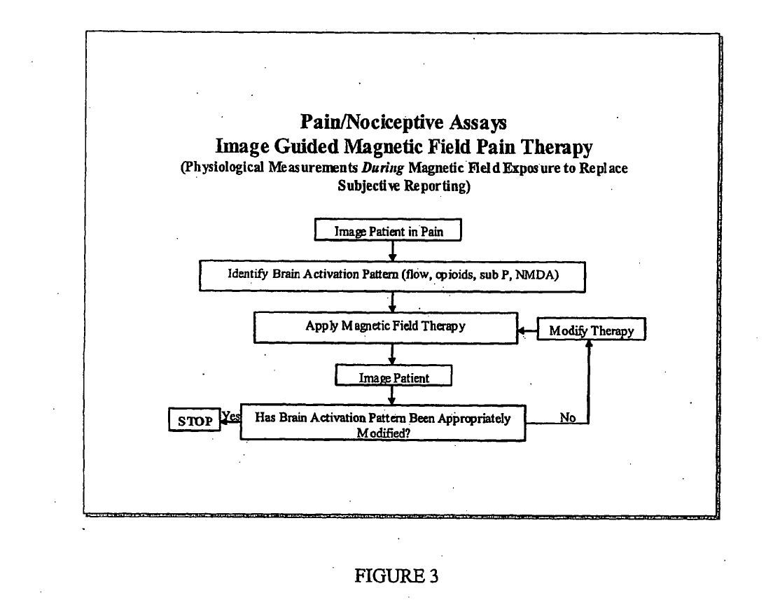

An embodiment of a method for the treatment of physiological, neurological and/or behavioral conditions is shown in the scheme of FIG. 3. First, an image of the brain of the patient in pain is taken and a brain activation pattern is identified (e.g., flow, opioids, substance-P, NMDA receptor). Second, a specific pulsed magnetic field is applied and other image of the brain of the patient is taken to verify whether the brain activation pattern has been appropriately modified. If modified sufficiently, then the method ceases. If not sufficiently modified, the steps are repeated. The specific pulsed magnetic field is applied again and an image of the brain is taken, and so on. The steps of applying the specific pulsed magnetic field and imaging may be simultaneous.

The method for treatment may be customized to individuals for the treatment of, for instance, neurological disorders or symptoms like pain, anxiety or depression permitting development and evaluation of treatment on an individual basis through the imaging of specific targets. Pulsed magnetic field parameters are preferably chosen to optimize their effectiveness in producing physiological, neurological and/or behavioral responses.

The method of treatment of the present invention may be applied to various areas of the body and should not be limited only to areas of the brain.

The method of the present invention may also be used as a tool for diagnosis. One embodiment of a method for the diagnosis of physiological, neurological and/or behavioral conditions includes a method for the diagnosis of a disease condition in a subject. The method involves exposing the subject to a specified pulsed magnetic field (Cnps) for a time effective to produce a physiological response. A physiological function is then monitored with a functional and/or molecular imaging device to evaluate and access the change in the selected physiological function to determine the disease condition, for instance, classifying the subject into a disease category. In preferred embodiments, BOLD fMRI (Blood Oxygen Level Dependent functional MRI) is used as the imaging device.

The specific pulsed magnetic field (Cnps) may be targeted to a specific target tissue of the subject, which is selected to affect a specific physiological function. The physiological function may be selected from the group consisting of a sensory function, motor function, and a cognitive function.

The method of diagnosis may be used to diagnose central nervous disorders such as pain, anxiety, or depression. It may also be used to diagnose a peripheral disorder such as rheumatoid- or osteo-arthritis, fibromyalgia, muscular dystrophy, and general pain.

Other embodiments of the invention are directed to the use of image-guided application of pulsed magnetic fields to diagnose physiological, neurological and/or behavioral pathologies or conditions and/or to the use of image-guided application of pulsed magnetic fields to treat physiological, neurological and/or behavioral pathologies or conditions. The use of image-guided application of pulsed magnetic fields to diagnose physiological, neurological and/or behavioral pathologies or conditions allows one to determine the severity of the pathology or condition.

Other potential uses of the present invention include, but are not limited to, other modes of functional imaging, treatment modalities, applications for use in veterinary medicine, horticultural, agricultural, entertainment purposes such as optimizing virtual reality or sensory modalities, psychogenicity, athletic performance enhancement, or image guided transcranial magnetic stimulation.

The above disclosure generally describes preferred embodiments of the present invention. A more complete understanding can be obtained by reference to the following specific EXAMPLES. These EXAMPLES are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Location of Pain Centers

Location of pain centers is important in discovering the cause of pain and in differential diagnosis. A patient with idiopathic pain (pain from an unknown origin) can be placed in an imaging device and baseline images are taken. The patient is exposed to a specific pulsed magnetic field (Cnp) previously shown to activate pain centers. The degree of activation of pain centers along with their location will provide differential diagnosis based on the pattern of activation observed (FIG. 1). This information guides the treatment and subsequent studies will determine the effectiveness of that treatment.

### Treatment of Claustrophobia

In 1991 (C. Kallon, Prevention 43(10), 39-43), it was estimated that patients suffering from anxiety, panic and claustrophobic attacks compromised the quality and efficiency of MRI examinations in an estimated 20% of all patient examinations and results in a loss of approximately $62.5 million (USD) annually in the United States alone. Specific pulsed magnetic fields (Cnps) to eliminate/attenuate claustrophobia or associated anxiety or emotional reaction have been designed and shown to be effective. Claustrophobic patients who were unable to complete an MRI imaging session in the past would now be treated with a Cnp prior to and during the session. This would allow the successful acquisition of the MRI images.

In addition, Cnp application may be image-guided. Once the Cnps are sufficiently effective to allow the patient to enter the MRI system, images of the claustrophobic activated regions of the brain would be made. Then the effectiveness of the Cnp to alleviate the claustrophobia may be optimized by changing the Cnp parameters and determining from the changes in the images, which combination of parameters would be most effective. These optimized parameters would be used during the remainder of the diagnostic imaging session.

### Image Guided Pain Therapy

Heterogeneity in response to pain therapy is well known. Although a general pulsed magnetic field for analgesia would be effective for pain reduction in most patients, improved pain control in individuals is achieved by customizing the treatment to the individual by using imaging methods. A symptomatic patient would enter the MRI device. A specified pulsed magnetic field would be applied using the MRI device's magnetic field gradients. If the pain centers associated with pain control are optimally activated or inhibited, as deduced from the image taken of the brain, then the pain pulse sequence used would be effective. If the pain centers are not optimally affected, as deduced from the image taken of the brain, then the parameters of the pulsed magnetic field are modified and the imaging repeated. In this iterative manner, the pulsed magnetic field parameters are optimized. On completion of this optimization, the patient is removed from the MRI device. The optimized pulse sequence is then programmed into a pain therapy device. If, after prolonged use, tolerance to the pulsed magnetic fields develops, the patient can return for a subsequent imaging session(s) and the pulsed magnetic field parameters altered. FIG. 3 shows a flow chart which generalizes this EXAMPLE.

FIGS. 2a and 2b show a specific pain paradigm for a Blood Oxygen Level Dependent (BOLD) fMRI study.

The principle behind the Blood Oxygen Level Dependent (BOLD) contrast in MRI is that the area of brain tissue activated in a specific tissue will experience an increase in local blood flow to that region. BOLD MRI detects the change in concentration of deoxyhemo-globin using a specific blood oxygen level sensitive imaging sequence.

Changes in signal observed in the BOLD sensitive MRI images are on the order of about 1-3%. Therefore, a series of averages is obtained in order to determine that a region of interest has been activated. To observe the brain activation for a particular stimulus, there must be a paradigm with a series of stimulus-on and stimulus-off iterations. The paradigm for the pain study will be described below.

The pain protocol involved the use of a hot pain stimulus on a subject's hand. The baseline temperature was 35° C., which was maintained for 35 seconds with a 5.5 second ramp up to 49° C. The heat stimulus of 49° C. was maintained for 10 seconds before ramping down to the baseline temperature of 35° C. in 5.5 seconds.

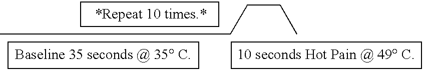

The pain paradigm shown above is synchronized with the image volume acquisition. Using a Gradient Echo EPI sequence, the entire brain volume is imaged in exactly 7 seconds. A total of 8 image volumes are collected per iteration of the pain paradigm for a total of 79 brain volumes (a total of 10 iterations was performed). The first 6 volumes are baseline and the last 2 volumes collected represent the pain stimulus.

FIG. 2a shows, as mentioned above, an increase in the activation of pain centers in the brain for an individual responding to a thermal stimulus on his or her non-dominant right hand. FIG. 2b shows the effect of applying a specific pulsed magnetic field, whereby there is a decrease in the activation of pain centers in the brain for the same individual shown in FIG. 2a responding to the same thermal stimulus.

The fMRI data collected (in FIGS. 1, 2a and 2b) is analyzed by using Statistical Parametric Mapping (SPM99) software. The software uses the a priori information from the paradigm design to compare the “expected” signal changes to the actual signal changes over the course of all 79-brain volumes acquired. This “expected” signal change is displayed in the top right-hand corner of the FIGURES.

The top left-hand corner of the FIGURE shows a “glass” brain, which is an “average” human brain created by the Montreal Neurological Institute from several hundred adult brains imaged. The SPM software aligns all of the data collected to this average brain so that brain regions of activation between multiple subjects can easily be compared. The glass brain displays all of the pixels, which are above a statistical threshold chosen by the user. The threshold for the pain experiments in FIGS. 2a and 2b was T=3.93. In the SPM software, it is possible to display the activated pixels shown in the glass brain on a set of 3 high resolution canonical images, as is seen in the bottom portion of the figures. The slice positions are defined in the glass brain by three arrows, one in each of the three planes (sagittal, coronal and axial), which correspond to the sagittal, coronal and axial images displayed in the lower left corner of the figure. For display purposes, slices were chosen that illustrate the most interesting regions of the brain activated, but more brain regions are activated than displayed in the high-resolution images.

### Image Guided Transcranial Magnetic Field Therapy

Affective disorders are a common and serious psychiatric/neurological clinical problem. Transcranial magnetic stimulation (TMS) has been as effective as electroconvulsive shock treatment but has significantly less risk and has been effective in drug-resistant patients. To date, TMS or repetitive TMS (rTMS) has not been image guided using functional and/or molecular imaging methods. A patient would be placed in an MRI device and a TMS coil would be placed on the patient's head. The volume of the brain targeted by the TMs coil would be determined by the measurement of induced current using current density magnetic resonance imaging. The TMs pulse, which is a high intensity pulse (approximately 10,000 T/s), would then be replaced with the specific pulsed magnetic field (Cnp). This would alter image contrast (as in EXAMPLE (1) and allow optimization of the pulse for the patient (as in EXAMPLE 3). Hence, the patient would then be treated acutely with rTMS and then maintained using the Cnp.

Although preferred embodiments of the invention have been described herein in detail, it will be understood by those skilled in the art that variations may be made thereto without departing from the spirit of the invention.

1. A method for treatment and/or diagnosis of a physiological, neurological and/or behavioral pathology or condition in a subject, the method comprising:
applying a pulsed magnetic field to a target area in the subject, in combination with imaging the targeted area to verify effectiveness of the pulsed magnetic field.

2. The method of claim 1, wherein the pulsed magnetic field is a specific pulsed magnetic field that functionally activates metabolic and molecular processes in the brain.

3. The method of claim 1, wherein the pulsed magnetic field is a specific pulsed magnetic field that functionally inhibits metabolic and molecular processes in the brain.

4. The method of claim 2, wherein the specific pulsed magnetic field functionally activates metabolic and molecular processes in the brain to diagnose physiological, neurological and/or behavior pathology or condition.

5. The method of claim 3, wherein the specific pulsed magnetic filed functionally inhibits metabolic and molecular processes in the brain to treat pain, anxiety or depression.

6. The method of claim 1, wherein the pulsed magnetic field is a specific low frequency pulsed magnetic field (Cnp).

7. The method of claim 6, wherein the specific low frequency pulsed magnetic filed (Cnp) has a plurality of intermittent waveforms.

8. The method of claim 1, wherein the method is used to monitor an effect of the magnetic field on the physiological, neurological and/or behavioral pathology or condition of the subject.

9. The method of claim 1, wherein the imaging is achieved using a molecular, a functional, and/or an anatomical medical imaging device.

10. The method of claim 9, wherein the imaging device is selected from the group consisting of a magnetic resonance imaging device, a positron emission tomography imaging device, and a single photon emission computerized tomography imaging device.

11. The method of claim 9, wherein the pulsed magnetic field is generated from the imaging device.

12. The method of claim 10, wherein the imaging device is the magnetic resonance imaging device (MRI) and the pulsed magnetic field is generated using the MRI.

13. The method of claim 12, wherein the MRI device is used to treat claustrophobia or anxiety while the subject is having a diagnostic imaging procedure.

14. The method of claim 1, wherein the method is used to select pulsed magnetic field parameters to optimize their effectiveness in producing physiological, neurological and/or behavioral responses.

15. The method of claim 1, wherein the pulsed magnetic field emphasizes image contrast.

16. The method of claim 1, wherein the method is for the diagnosis of the physiological, neurological and/or behavioral pathology or condition in the subject, wherein the application of the pulsed magnetic field to the targeted area in the subject initiates a physiological, neurological and/or behavioral response and the imaging of the targeted area permits monitoring of a physiological, neurological and/or behavioral function to determine the physiological, neurological and/or behavioral pathology or condition of the subject.

17. The method of claim 1, wherein both the application of the pulsed magnetic field and the imaging of the targeted area is done simultaneously.

18. The method of claim 16, wherein the pulsed magnetic field is a specific low frequency pulsed magnetic field (Cnp).

19. The method of claim 1, wherein the method is for the diagnosis of a disease condition in the subject, wherein the subject is within an imaging device and the application of the pulsed magnetic field to the targeted area in the subject is for a time effective to produce a physiological response and the imaging of the targeted area permits monitoring of a selected physiological function, the method further comprising:
evaluating a change in the selected physiological function with imaging of the targeted area;
assessing the change in the selected physiological function with imaging of the targeted area; and
classifying the subject into a disease category based on the assessment of the change in the selected physiological function.

20. The method of claim 19, wherein the subject is exposed to the pulsed magnetic field and imaging simultaneously while monitoring the selected physiological function.

21. The method of claim 19, wherein the imaging device is a functional and/or molecular imaging device.

22. The method of claim 19, wherein the pulsed magnetic field is a specific low frequency pulsed magnetic field (Cnp).

23. A method for treatment of a physiological, neurological and/or behavioral pathology or condition in a subject, the method comprising:
applying a pulsed magnetic field to a targeted area in the subject, in combination with imaging the targeted area to verify effectiveness of the pulsed magnetic field and
repeating application of the pulsed magnetic field and imaging until sufficient treatment of the pathology or condition is attained.

24. The method of claim 23, wherein both the application of the pulsed magnetic field and the imaging of the target tissue is are done simultaneously.

25. The method of claim 23, wherein prior to applying the pulsed magnetic field to the targeted area, the targeted area of the subject is imaged and an activation pattern of the targeted area is identified.

26. The method of claims 23, wherein the pulsed magnetic field is a specific low frequency pulsed magnetic field (Cnp).

27. The method of claim 1, wherein the method is for the treatment of the physiological, neurological and/or behavioral pathology or condition in the subject, the method further comprising:
optimizing the pulsed magnetic field based on the image; and
repeating the application of the optimized pulsed magnetic field and imaging until sufficient treatment of the condition is attained.

28. The method of claim 27, wherein both the application of the pulsed magnetic field and the imaging of the targeted area is done simultaneously.

29. The method of claim 27, wherein prior to applying the pulsed magnetic field to the targeted area, the targeted area of the subject is imaged and an activation pattern of the targeted area is identified.

30. The method of claim 27, wherein the pulsed magnetic field is a specific low frequency pulsed magnetic field (Cnp).

31. An electrotherapy apparatus for treatment and/or diagnosis of a physiological, neurological and/or behavioral pathology or condition in a subject, the apparatus comprising an imaging device and at least one pulsed magnetic field generating member, wherein the apparatus provides application of a pulsed magnetic field from the at least one pulsed magnetic field generating member to a targeted area in the subject, in combination with imaging the targeted area with the imaging device, to verify the effectiveness of the pulsed magnetic field.

32. The apparatus of claim 31, wherein the at least one pulsed magnetic field generating member is a tube and/or coil.

33. The apparatus of claim 32, wherein the tube and/or coil are a gradient tube and/or gradient coil.

34. The apparatus of claim 31, wherein the pulsed magnetic field is a specific pulsed magnetic field that functionally activates metabolic and molecular processes in the brain.

35. The apparatus of claim 31, wherein the pulsed magnetic field is a specific pulsed magnetic field that functionally inhibits metabolic and molecular processes in the brain.

36. The apparatus of claim 34, wherein the specific pulsed magnetic field functionally activates metabolic and molecular processes in the brain to diagnose physiological, neurologicaland/or behavioral pathology or condition.

37. The apparatus of claim 35, wherein the specific pulsed magnetic field functionally inhibits metabolic and molecular processes in the brain to treat pain, anxiety or depression.

38. The apparatus of claim 31, wherein the pulsed magnetic field is a specific low frequency pulsed magnetic field (Cnp).

39. The apparatus of claim 31, wherein the specific low frequency pulsed magnetic field (Cnp) has a plurality of intermittent waveforms.

40. The apparatus of claims 31, wherein the pulsed magnetic field is used to monitor an effect of the magnetic field on the physiological, neurological and/or behavioral pathology or condition of the subject.

41. The apparatus of claim 31, wherein the pulsed magnetic field is generated using the imaging device.

42. The apparatus of claim 31, wherein the imaging device is a molecular, a functional, and/or an anatomical medical imaging device.

43. The apparatus of claims 31, wherein the imaging device is selected from the group consisting of a magnetic resonance imaging device, a positron emission tomography imaging device, and a single photon emission computerized tomography imaging device.

44. The apparatus of claim 31, wherein the application of the pulsed magnetic field is used to select pulsed magnetic field parameters to optimize their effectiveness in producing physiological, neurological and/or behavioral responses.

45. The apparatus of claim 31, wherein the apparatus is for the diagnosis of the physiological, neurological and/or behavioral pathology or condition in the subject, wherein the pulsed magnetic field from the at least one pulsed magnetic field generating member initiates a physiological, neurological and/or behavioral response and the imaging device images the targeted area to permit monitoring of a physiological, neurological and/or behavioral function in order to determine the physiological, neurological and/or behavioral pathology or condition of the subject.

46. The apparatus of claim 45, wherein both the application of the pulsed magnetic field and the imaging of the targeted area is done simultaneously.

47. The apparatus of claim 31, wherein the apparatus is for the diagnosis of a disease condition in the subject within the imaging device, wherein the pulsed magnetic field from the at least one pulsed magnetic field generating member is applied to the subject for a time effective to produce a physiological response and the imaging device of the targeted area permits monitoring of a selected physiological function, evaluating and possessing a change in a selected physiological function in order to classify the subject into a disease category based on the assessment of the change in the selected physiological function.

48. The apparatus of claim 31, wherein the apparatus is for the treatment of the physiological, neurological and/or behavioral pathology or condition in the subject, wherein the apparatus applies the pulsed magnetic field and imaging until sufficient treatment of the pathology or condition is attained.

49. The apparatus of claim 48, wherein the imaging device is capable of imaging the targeted area of the subject, initially, so that an activation pattern of the targeted area is identified.

50. The apparatus of claim 31, wherein the apparatus is for the treatment of the physiological, neurological and/or behavioral pathology or condition in the subject, wherein the pulsed magnetic field from the at least one pulsed magnetic field generating member is optimized based on the image and the application of the optimized pulsed magnetic field and imaging is repeated until sufficient treatment of the condition is attained.

51. The apparatus of claim 50, wherein the imaging device is capable of imaging the targeted area of the subject, initially, so that an activation pattern of the targeted area is identified.
